(19) United States
(12) Patent Application Publication
(10) Pub. No.: US 2020/0163642 A1
(43) Pub. Date: May 28, 2020

(54) METHODS FOR COMPUTATIONAL MODELING TO GUIDE INTRATUMORAL THERAPY

(71) Applicant: University of Vermont and State Agricultural College, Burlington, VT (US)

(72) Inventors: C. Matthew KINSEY, Williston, VT (US); Jason H.T. BATES, S. Burlington, VT (US)

(21) Appl. No.: 16/637,668

(22) PCT Filed: Aug. 8, 2018
(86) PCT No.: PCT/US18/45884

§ 371 Date: Feb. 7, 2020

Related U.S. Application Data
(60) Provisional application No. 62/542,623, filed on Aug. 8, 2017.

(51) Int. Cl. A61B 6/00; A61B 6/03; G06T 7/00; G06T 17/00; G16H 30/40; G16H 50/50; G16H 20/17

(52) U.S. Cl. CPC A61B 6/5217; A61B 6/032; A61B 6/507; A61B 6/461; A61B 8/5223; G06T 17/00; G16H 30/40; G16H 50/50; G16H 20/17; G06T 7/0014

(57) ABSTRACT

Methods are presented for simulating drug movement within a model of a tumor that is mapped to the specific anatomy of the corresponding tumor in a patient as determined by imaging. With a segmentation of the tumor into distinct interconnected compartments and pre-determined initial parameters for the distributed tissue diffusivities and perfusion levels, the disclosed techniques can be used to predict the drug concentration throughout the tumor as a function of time, as well as the accumulation of drug in the rest of the body. In this way, advantageous initial parameters may be determined using the model. It is also possible to predict drug concentration throughout the tumor for a given intravenous injection of drug. Such a model serves an important role in treatment planning for lung tumors.

### CROSS-REFERENCE TO RELATED APPLICATIONS

This application claims priority to U.S. Provisional Application No. 62/542,623, filed on Aug. 8, 2017, now pending, the disclosure of which is incorporated herein by reference.

### STATEMENT REGARDING FEDERALLY SPONSORED RESEARCH

This invention was made with government support under contract nos. R01 HL-124052, R01 HL-130847, and K23 HL-133476, each awarded by the National Institutes of Health. The government has certain rights in the invention.

### FIELD OF THE DISCLOSURE

The present disclosure relates to guiding the injection of drugs for intratumoral therapy using computer modeling.

### BACKGROUND OF THE DISCLOSURE

Intratumoral therapy, for example, for lung cancer, is typically performed with the same dose of drug applied in the same manner for all tumors, and with little guidance regarding how and where to inject, regardless of tumor size or other characteristics. In this way, the injection strategy is entirely empirical.

Injection of drugs, for example, chemotherapeutic drugs, directly into lung tumors using endo-bronchial ultrasound guidance or direct visualization, or via percutaneous approaches, has the potential to increase the treatment effects on the tumor while significantly reducing systemic side-effects. In order to achieve full efficacy, however, the injected drug must reach a given minimal concentration in all regions of the tumor.

Accordingly, there is a long felt need for an improved injection strategy to increase the treatment effects on the tumor while significantly reducing systemic side-effects.

### BRIEF SUMMARY OF THE DISCLOSURE

Methods are presented for simulating drug movement within a model of a tumor that is mapped to the specific anatomy of the corresponding tumor in a patient as determined by imaging. With a segmentation of the tumor into distinct interconnected compartments and pre-determined initial parameters for the distributed tissue diffusivities and perfusion levels, the disclosed techniques can be used to predict the drug concentration throughout the tumor as a function of time, as well as the accumulation of drug in the rest of the body. In this way, advantageous initial parameters may be determined using the model. It is also possible to predict drug concentration throughout the tumor for a given intravenous injection of drug. Such a model serves an important role in treatment planning for lung tumors.

### DESCRIPTION OF THE DRAWINGS

For a fuller understanding of the nature and methods of the disclosure, reference should be made to the following detailed description taken in conjunction with the accompanying drawings, in which:

FIG. 1A is an exemplary series of drug concentration diagrams, where each diagram shows, in two dimensions, how an injected drug diffuses through tumor tissue, and the series shows the evolution over a period of 100 arbitrary time units;

FIG. 1B is an exemplary series of drug concentration diagrams, where each diagram shows, in two dimensions, how an injected drug diffuses through tumor vasculature, and the series shows the evolution over a period of 100 arbitrary time units; and

FIG. 2 is a flow chart of an embodiment of the present disclosure.

### DETAILED DESCRIPTION OF THE DISCLOSURE

Injection strategies (i.e., locations and doses of drug) to achieve the objective of full efficacy are not easily identified because they are strongly influenced by the diffusivity of the agent within the tumor tissue and by the tumor perfusion that acts to clear the agent and distribute it to the rest of the body. The present disclosure provides techniques utilizing a computational model to takes tumor anatomy, vasculature, and biophysical properties into account and provide guidance for improved injection strategies. Reference is made to a non-limiting, two-dimensional version of such a model, which was developed to demonstrate proof of concept and to illustrate embodiments of the disclosure.

Methods incorporating the present computational techniques may provide guidance for intratumoral injection of a drug (such as chemotherapy, immunotherapy, etc.) For example, methods may be used to determine advantageous location(s) of injection, delivery device (such as a single hollow needle), delivery rate, and/or dose for delivery of an agent based on characteristics of a subject tumor. Such characteristics may include, for example, tissue and blood vessel density.

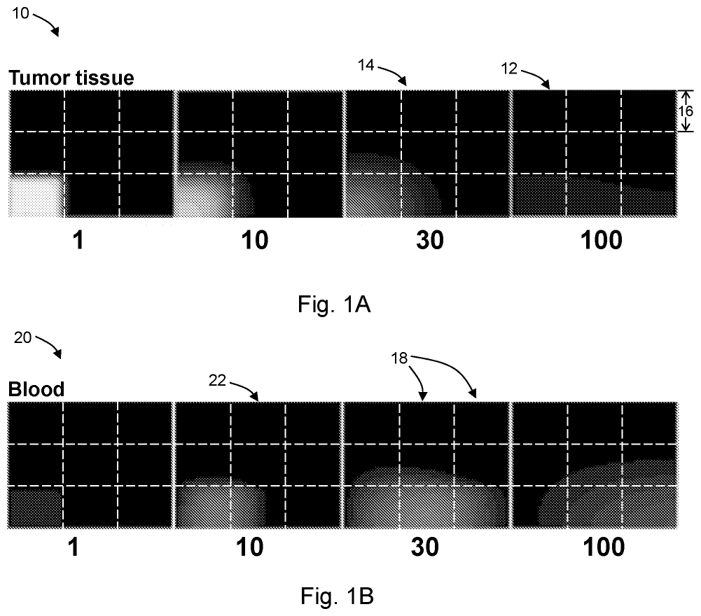

In embodiments of the present disclosure, a model of a tumor may be mapped to the specific anatomy of a tumor in a patient as determined by imaging. Such a model may be two-dimensional (2D) or three-dimensional (3D), and imaging may be obtained accordingly. The tumor is modeled as a plurality of compartments. In a simplified, two-dimensional example, a lung tumor is modeled as a rectangular region divided into multiple rectangular compartments of equal area (see, FIG. 1A). The compartments may be of equal size or otherwise.

Movement of a drug (direction and magnitude) through the tumor is modeled based on the compartment types. For example, movement of a drug between adjacent tissue compartments 12 may be modeled as diffusion according to the difference in the respective concentrations of drug in the compartments, an intrinsic tumor diffusivity coefficient (D), and the size (e.g., length 16 (2D models) or area (3D models)) of the shared boundary 14 between the compartments. Tumors may be pervaded throughout by a microvasculature represented by a series of vasculature compartments 22 that are geographically congruent with but physically distinct from the tissue compartments. Drug movement between a tissue compartment 12 and into a vasculature compartment may be modeled as diffusing at a rate proportional to D, the length (or area for 3D models) over which the tissue and vascular compartments intersect, and the drug concentration difference between tissue and vasculature compartments. Drug movement between adjacent vasculature compartments 22 may be modeled as convection according to the direction and magnitude of the blood flow between them. The model may have initial conditions (parameters) representing, for example, the location and amount of drug immediately following its injection into the tumor. In some embodiments, the compartments have the same size within each classification, but different size between classifications.

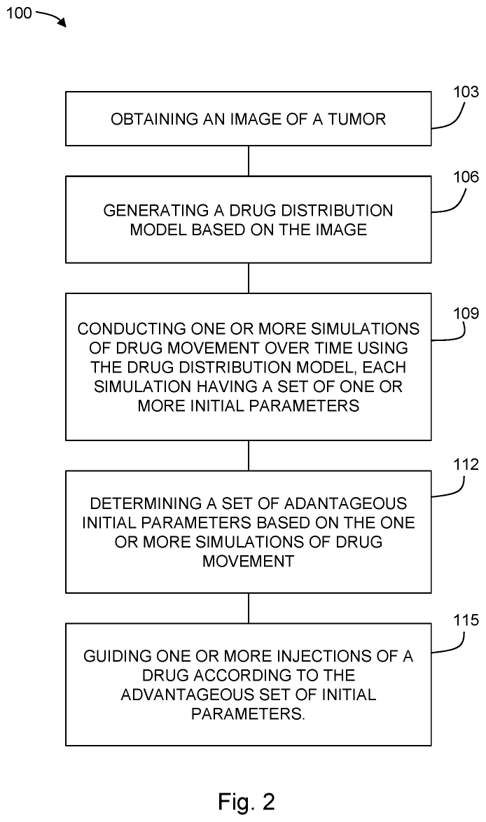

With reference to FIG. 2, the present disclosure may be embodied as a method 100 for intratumoral drug treatment is provided. The method 100 includes obtaining 103 an image of a tumor. The image may be a 2D image. In other embodiments, the image is a 3D image. Such images are known in the art and can be captured using any imaging modality such as, for example, computer-tomography (CT) imaging. It should be noted that the term 3D image is used herein to describe data that can be used to generate 3D imaging information. For example, the 3D image may be a set of 2D images, such as, for example, a set of image slices. As such, the image may be obtained 103 using a computed tomography scanner or other type of device (e.g., MRI, PET-CT, etc.) In some embodiments, the image is obtained 103 by retrieval from an electronic storage device. For example, the electronic storage device may be a disk drive, a flash drive, an optical drive, or any other type of memory. Such an electronic storage device may be local or remote (e.g., having an intervening network). The method 100 further includes generating 106 a drug distribution model based on the obtained 103 image. The model may be generated 106 by dividing the tumor (tumor image) into a plurality of compartments. In 2D embodiments, each compartment of the plurality of compartments may be a regular shape such as, for example, rectangular. In 3D embodiments, each compartment of the plurality of compartments may be a regular shape such as, for example, a cuboid. In this way, each compartment has one or more adjacent compartments with a shared boundary between each adjacent compartment.

In the model, a classification is assigned to each compartment based on one or more characteristics of the portion of the tumor making up the compartment. For example, each compartment may be classified as either a tissue compartment or a vasculature compartment. As described above, drug movement between each pair of adjacent compartments within the model may be determined according to the classification of the respective compartments.

The method 100 further includes conducting 109 one or more simulations of drug movement over time using the drug distribution model. Each simulation is conducted 109 by setting a set of one or more initial parameters. Such initial parameters may include, for example, one or more injection location(s), a delivery modality (e.g., size and/or type of needle, etc.), a delivery rate, a delivery dose, tissue diffusivity, tissue perfusion, and/or other parameters. Where more than one injection location is provided, other parameters may have a value for each injection location. For example, a delivery dose may be provided for each injection location, and each provided delivery dose may be the same as or different from the other delivery dose(s). The simulation is allowed to run by calculating the drug movement between each pair of adjacent compartments (for example, magnitude and direction) over time using the applicable classification model for each compartment and adjacent compartments. In this way, the model may show, for example, for a given load and location of drug within the tumor, a drug concentration within each compartment over time, etc. Each simulation may be run with differing initial parameters. In this way, the resulting outcomes of each simulation may be evaluated.

In some embodiments, drug movement over a period of time by subdividing the period of time into discrete units of time. In this way, the drug movement over a period of 100 units is the accumulated movement of the drug over each unit period of time for the 100 units. The units of time may be arbitrary. The units of time may be selected according to the units of the initial parameters (e.g., where diffusivity is provided in terms of seconds, etc.)

The method 100 further includes determining 112 a set of advantageous initial parameters based on the one or more simulations. A set of initial parameters may be determined to be advantageous by evaluating the outcome of each conducted 109 simulation according to any criteria as will be apparent to one of skill in the art. For example, a criterion may be shortest time to achieve a drug concentration representing full efficacy throughout the tumor. In some embodiments, multiple criteria may be used such as, for example, the shortest time for the least number of injection locations. In another example, a criterion may be the minimization of drug accumulation in the patient outside of the tumor. Other criteria will be apparent in light of the present disclosure. The advantageous set of initial parameters may correspond with the initial parameters of the most advantageous simulation.

The method 100 may further include guiding 115 one or more injections of a drug according to the advantageous set of initial parameters. For example, an actuator may be used to position a needle for injection of the drug into the tumor. In another embodiment, an actuator may be used to guide the position of a syringe which is manually advanced by a medical practitioner.

The tumor may be homogenous, heterogenous, or may have varying degrees of homogeneity, and therefore, the model may be constructed as such. The tumor (and therefore, the model) may be substantially isotropic or non-isotropic.

In some embodiments, the magnitude and direction of drug movement over time between tissue compartments 12 is based on a difference in drug concentration between each compartment, a tumor diffusivity coefficient, and a length 16 (or area in the case of a three-dimensional model) of the shared boundary 14 between each compartment.

In some embodiments, the magnitude and direction of drug movement over time between a tissue compartment 12 and a vasculature compartment 22 is based on a tumor diffusivity coefficient of the tissue compartment 12, a length 16 (or area) of the shared boundary between the tissue compartment 12 and the vasculature compartment 22, and a difference in drug concentration between the tissue compartment 12 and the vasculature compartment 22.

In some embodiments, the magnitude and direction of drug movement over time between vascular compartments 22 is based on a direction of blood flow and a magnitude of blood flow.

In some embodiments, the plurality of compartments are of equal size.

In some embodiments, the one or more tissue compartments 12 are of equal size.

In some embodiments, the one or more vasculature compartments 22 are of equal size.

In some embodiments, the method for intratumoral drug treatment also includes displaying the one or more recommended injection points to a practitioner.

In some embodiments, the one or more recommended injection points are displayed as a heat map.

In some embodiments, the method for intratumoral drug treatment also includes obtaining the image of the tumor via computed tomography (CT) scan. In some embodiments, the method for intratumoral drug treatment also includes obtaining the image of the tumor via positron emission tomography-computed tomography (PET-CT) scan. In some embodiments, the method for intratumoral drug treatment also includes obtaining the image of the tumor via magnetic resonance imaging (MRI). In some embodiments, the method for intratumoral drug treatment also includes obtaining the image of the tumor via ultrasound, either endobronchial or surface. Other imaging modalities or combinations of modalities may be used to obtain suitable images for use with the presently disclosed technique.

In some embodiments, the method for intratumoral drug treatment also includes physically guiding injection of a drug with a mechanical apparatus.

In some embodiments, the method for intratumoral drug treatment also includes determining a drug delivery device based on the one or more simulations of drug movement over time using the drug distribution model. The drug delivery device may be a single hollow needle.

In some embodiments, the method for intratumoral drug treatment also includes determining a drug delivery rate based on the one or more simulations of drug movement over time using the drug distribution model.

In some embodiments, the method for intratumoral drug treatment also includes determining a drug delivery dose based on the one or more simulations of drug movement over time using the drug distribution model.

In another aspect of the present disclosure, a method for intratumoral drug treatment is provided. The method includes obtaining a series of images of a tumor. The method further includes generating a three-dimensional drug distribution model based on the series of images, wherein the model comprises a plurality of rectangular prismatic compartments, wherein each compartment is classified as either a tissue compartment or a vasculature compartment, and wherein each compartment has one or more adjacent compartments with a shared boundary between each adjacent compartment. The method further includes conducting one or more simulations of drug movement over time using the drug distribution model, each simulation having set of one or more initial parameters, wherein the drug movement between each pair of adjacent compartments has a magnitude and a direction. The method further includes determining one or more recommended injection points based on the one or more simulations of drug movement over time using the drug distribution model.

### Results of Experimental Embodiment

FIGS. 1A and 1B show how the drug first diffuses within the tumor tissue from its early configuration shortly after injection (1 time unit) and then begins to make its way into the tumor vasculature (10 time units). The figures show how, as the drug becomes more diffuse throughout the tissue (FIG. 1A), the drug in the blood (vasculature) (FIG. 1B) moves to the right as it is transported by bulk convection (30 time units) until eventually it has been transported largely to the rest of the body (100 time units).

Although the present disclosure has been described with respect to one or more particular embodiments, it will be understood that other embodiments of the present disclosure may be made without departing from the spirit and scope of the present disclosure.

What is claimed is:

1. A method for intratumoral drug treatment, comprising:
obtaining an electronic image of a tumor;
generating a drug distribution model based on the image, the model comprising a plurality of compartments, each compartment being classified as either a tissue compartment or a vasculature compartment based on the obtained image, and wherein each compartment has one or more adjacent compartments with a shared boundary between the compartment and each adjacent compartment;
conducting one or more simulations of drug movement over time using the drug distribution model, each simulation having a set of one or more initial parameters, wherein the drug movement between each pair of adjacent compartments has a magnitude and a direction according to the classifications of the respective compartments; and
determining a set of one or more advantageous initial parameters based on the one or more simulations.

2. The method of claim 1, wherein the magnitude and direction of drug movement between adjacent tissue compartments is based on a difference in drug concentration between each compartment, a tumor diffusivity coefficient, and a size of the shared boundary between each compartment.

3. The method of claim 1, wherein the magnitude and direction of drug movement between a tissue compartment and an adjacent vasculature compartment is based on a tumor diffusivity coefficient of the tissue compartment, a size of the shared boundary between the tissue compartment and the vasculature compartment, and a difference in drug concentration between the tissue compartment and the vasculature compartment.

4. The method of claim 1, wherein the magnitude and direction of drug movement between adjacent vascular compartments is based on a direction of blood flow and a magnitude of blood flow.

5. The method of claim 1, wherein each compartment of the plurality of compartments is rectangular.

6. The method of claim 1, wherein the image is a three-dimensional image and the drug distribution model is a three-dimensional model.

7. The method of claim 6, wherein the imaging is a set of images.

8. The method of claim 7, wherein the set of images is a set of image slices.

9. The method of claim 8, wherein the set of images is obtained using computed tomography.

10. The method of claim 6, wherein each compartment of the plurality of compartments is cuboid.

11. The method of claim 1, wherein the plurality of compartments are of equal size.

12. The method of claim 1, wherein the one or more tissue compartments are of equal size.

13. The method of claim 1, wherein the one or more vasculature compartments are of equal size.

14. The method of claim 1, wherein the set of advantageous initial parameters comprises one or more injection locations.

15. The method of claim 14, further comprising displaying the one or more injection locations of the set of advantageous initial parameters using a display.

16. The method of claim 15, wherein the one or more injection points are displayed as a heat map.

17. The method of claim 1, wherein the set of advantageous initial parameters comprises a drug delivery modality.

18. The method of claim 1, wherein the set of advantageous initial parameters comprises a drug delivery rate.

19. The method of claim 1, wherein the set of advantageous initial parameters comprises a drug delivery dose.

20. The method of claim 1, wherein the image is obtained by retrieval from an electronic storage device.

21. The method of claim 1, further comprising guiding one or more injections of a drug according to the advantageous set of initial parameters.
